(19) United States
(12) Patent Application Publication
(10) Pub. No.: US 2007/0260072 A1
(43) Pub. Date: Nov. 8, 2007

(54) Concurrent Sulfur Dioxide Oxidation Process and its Use in Manufacture of Tetrabromophthalic Anhydride

(75) Inventors: William B. Harrod, Minden, LA (US); Tyson J. Hall, Magnolia, AR (US); Christopher S. Knight, Prairieville, LA (US); John C. Prindle, Baton Rouge, LA (US); David M. Armstrong, Walker, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/568,308

(22) PCT Filed: Apr. 27, 2004
(86) PCT No.: PCT/US04/12861

§ 371 Date: Oct. 25, 2006

(51) Int. Cl. C07D 307/89; C01B 17/79

(52) U.S. Cl. 549/247; 423/533

(57) ABSTRACT

Sulfur trioxide is formed by a process wherein a first gaseous stream comprised of SO₂, SO₃, and oxygen and/or air is passed into a bed of a vanadium-containing catalyst that oxidizes S0₂ to SO₃and that releases therefrom a second gaseous stream comprised of sulfur trioxide. This process is improved in a first case by providing vaporized sulfur in the first gaseous stream so that the resultant mixture passes through a substantial portion of the catalyst bed, and maintaining the catalyst bed at one or more temperatures in the range of about 450 to about 700° C. The sulfur is oxidized to S0₂. As a result, the second gaseous stream released from the downstream end portion of the catalyst bed has an enriched content of sulfur trioxide, which can be used for production of compounds such as tetrabromophthalic anhydride. In a second case, a stream of sulfur dioxide is generated from sulfur and an oxidant, and this stream is introduced into the first gaseous stream referred to above. In this second case, the feed of sulfur dioxide replaces the vaporized sulfur used in the first case. As in the first case, an enriched stream of sulfur trioxide is released from the downstream end of the catalyst and can be used for producing compounds such as tetrabromophthalic anhydride.

### TECHNICAL FIELD

This invention relates to improved process technology pertaining to oxidation of sulfur dioxide to sulfur trioxide, and to improving operations in which oxidation of sulfur dioxide to sulfur trioxide is involved.

### BACKGROUND

The oxidation of sulfur dioxide to sulfur trioxide using oxygen or air and a suitable catalyst such as vanadium pentoxide is well known. Such an oxidation step is typically included in the contact process for producing sulfuric acid. Also, passing a gaseous stream containing sulfur dioxide, air and some sulfur trioxide through a bed of a vanadium-containing sulfuric acid catalyst such as preferably used in the practice of this invention maintained at about 824-1100° F. (ca. 440-593° C.) to oxidize sulfur dioxide to sulfur trioxide has been carried out heretofore. Further, it is known that sulfur can be oxidized into sulfur dioxide using a suitable oxidant such as air (auto ignition 261° C.) or oxygen (at less than 260° C.). However, the conversion of sulfur dioxide into sulfur trioxide requires a temperature activated catalyst such as a vanadium-containing catalyst, e.g., vanadium pentoxide or the like.

In Latimer and Hildebrand, The Reference Book of Inorganic Chemistry, revised edition, copyrighted in 1940 by The Macmillan Company, New York, it is stated on page 246 with reference to the contact process that “One of the factors in the successful operation of the process is the removal from the sulfur dioxide of all impurities which may ‘poison’ the catalyst and render it inactive. It is particularly important to remove traces of solid sulfur, selenium dioxide, mercury, and compounds of phosphorus and arsenic.”

### BRIEF SUMMARY OF THE INVENTION

In one of its embodiments this invention provides an improved process in which a gaseous stream comprised of sulfur dioxide, sulfur trioxide, and oxygen and/or air is passed through and in contact with a bed of a vanadium-containing catalyst such as a vanadium oxide catalyst (typically vanadium pentoxide), and preferably a bed of a mixture of complex inorganic salts (oxosulfato vanadates) containing sodium, potassium and vanadium salts on crystalline silica support, or a catalyst including silica as a support within a salt mixture comprised of potassium and/or cesium sulfates, and vanadium sulfates coated on the solid silica support, that oxidizes sulfur dioxide to sulfur trioxide and that releases therefrom a product gaseous stream comprised of sulfur trioxide. In this embodiment the improvement comprises having molten sulfur come into contact with the catalyst and maintaining the catalyst bed at one or more temperatures at which (i) sulfur coming into contact with the catalyst is vaporized before the gaseous product(s) formed therefrom are released from a downstream end portion of the catalyst bed and (ii) the gaseous stream released from the downstream end of said bed has an enriched content of sulfur trioxide.

The temperatures of the catalyst bed bring about (i) and (ii) above differ from each other to some extent. In order to oxidize the sulfur dioxide to sulfur trioxide as in (ii) above the vanadium-containing catalyst bed should be at one or more temperatures in the range of about 450 to about 700° C., and preferably in the range of about 450 to about 600° C. However to vaporize sulfur as in (i) above, one or more temperatures in the range of about 300 to about 450° C. are sufficient although one or more temperatures in the range of about 300 to about 700° C. can be used. Thus in conducting the above embodiment of this invention:
a) the catalyst bed can be at one or more temperatures in the range of about 450 to about 700° C. or preferably at one or more temperatures in the range of about 450 to about 600° C.;
b) the catalyst bed can have two or more stages at different temperatures, such as for example an upstream portion at one or more temperatures in the range of about 300 to below about 450° C. to vaporize the sulfur and also to result in some oxidation of sulfur by SO₃ to SO₂, and a more downstream portion at one or more temperatures in the range of about 450 to about 700° C. or preferably at one or more temperatures in the range of about 450 to about 600° C. to cause the oxidation of SO₂ to SO₃ to take place therein; or
c) at least two reactors can be arranged in tandem with a first reactor being provided with, e.g., internal packing surfaces or other inert surfaces that are at one or more temperatures sufficient to vaporize the sulfur, such as at one or more temperatures in the range of about about 300 to about 700° C., preferably in the range of about 300 to about 450° C., and more preferably in the range of about 300 to about 350° C. Because the incoming gaseous stream to such first reactor also contains SO₃, at least some oxidation of sulfur by SO₃ to SO₂ is likely to occur in such first reactor. A second reactor in this arrangement contains the above bed of vanadium-containing catalyst that oxidizes sulfur dioxide to sulfur trioxide, which bed is maintained at one or more temperatures in the range of about 450 to about 700° C. or preferably at one or more temperatures in the range of about 450 to about 600° C. to cause the oxidation of sulfur dioxide to sulfur trioxide to take place therein.
   Of these alternatives, a) is preferred as it is the simplest to practice, and b) and c) tend to be more costly.

One of the features of the above embodiment of this invention when alternative a) is employed is that because of the high temperature(s) at which the catalyst bed is operated, the sulfur is vaporized as it comes into contact with the catalyst bed. This enables the vapors to be subjected to oxidation as they pass through the catalyst bed so that the gaseous stream released from the downstream end of catalyst bed has an enriched content of sulfur trioxide. In addition, at the high temperature(s) at which the catalyst bed is operated, sulfur vaporizes to such an extent that unduly rapid formation and buildup of sulfur coatings or deposits on the catalyst surfaces does not occur. Thus the catalytic activity of the catalyst in the bed is not adversely affected.

As will be seen hereinafter, the improved process technology of this invention can be effectively utilized for various purposes wherein sulfur trioxide is put to use.

Another embodiment of this invention is an improvement in a process in which a first gaseous stream comprised of sulfur dioxide, sulfur trioxide and oxygen and/or air is passed into a bed of a vanadium-containing catalyst that oxidizes sulfur dioxide to sulfur trioxide and that releases therefrom a product gaseous stream comprised of sulfur trioxide. In this embodiment the improvement comprises oxidizing sulfur with air, oxygen and/or sulfur trioxide (preferably with a gaseous stream which contains (i) at least sulfur trioxide and air or oxygen, or (ii) sulfur trioxide, air and added oxygen) to form a second gaseous stream enriched in sulfur dioxide and introducing at least a portion of the second gaseous stream into the first gaseous stream to form a mixed gaseous stream, and passing the mixed gaseous stream into an upstream portion of the above catalyst bed maintained at one or more temperatures in the range of about 450 to about 700° C., and preferably in the range of about 450 to about 600° C. This results in the formation of a product stream emanating from a downstream portion of the catalyst bed that is enriched in sulfur trioxide. The amount of sulfur trioxide in the product stream tends to be greater than could be predicted from the oxidation of the total amount of sulfur dioxide in the mixed gaseous stream. The oxidation of sulfur in this embodiment of the invention is carried out in a separate reactor which feeds its effluent stream as a side stream into the first gaseous stream. This reactor is not an inline reactor. In conducting this embodiment of the invention the first gaseous stream need not contain sulfur trioxide if sulfur trioxide is used in the oxidation of the sulfur in such separate reactor and if an excess amount of sulfur trioxide is fed into the separate reactor so that the feed to the first gaseous stream from the separate reactor contains some residual sulfur trioxide. However, it is preferred that the first gaseous stream and the feed to the first gaseous stream from the separate reactor both contain sulfur trioxide as this tends to further increase the amount of sulfur trioxide emanating from the vanadium-containing catalyst bed over and above that which could be predicted from the sum of (A) the amount of sulfur trioxide formed by direct mole-for-mole oxidation of sulfur dioxide to sulfur trioxide and (B) the total amount of sulfur trioxide present in the first gaseous stream and in the feed to the first gaseous stream from the separate reactor, assuming all such sulfur trioxide passed through the catalyst bed unchanged.

In the various embodiments of this invention the catalyst used is preferably a fixed bed of a vanadium-containing catalyst that oxidizes sulfur dioxide to sulfur trioxide and that releases therefrom a product gaseous stream comprised of sulfur trioxide.

Other embodiments, features and advantages of this invention will be still further apparent from the ensuing description, appended claims and accompanying drawings.

### BRIEF DESCRIPTION OF THE DRAWINGS

FIG. 1 is a schematic flow diagram of a preferred way pursuant to this invention of carrying out a process of producing SO₃ from a gaseous stream of SO₂ containing oxygen and/or air and a minor amount of SO₃.

FIG. 2 is a schematic side view diagram of a reactor in which the sequential reactions of equations (1) and (2) above can be carried out.

FIG. 3 is a schematic flow diagram of a preferred way of utilizing a process of this invention in forming oleum and using the oleum as a reaction medium in the preparation of a commercially important flame retardant.

FIG. 4 is a schematic flow diagram of another way pursuant to this invention of carrying out a process of producing SO₃ from a gaseous stream of SO₂ containing oxygen and/or air and preferably a minor amount of SO₃ wherein a side stream comprised of system-generated sulfur dioxide is fed into a system such as that of FIG. 1 but without feeding sulfur into the system as in FIG. 1.

FIG. 5 is a schematic flow diagram of laboratory apparatus used in developing information and data useful in scale up of process technology of this invention.

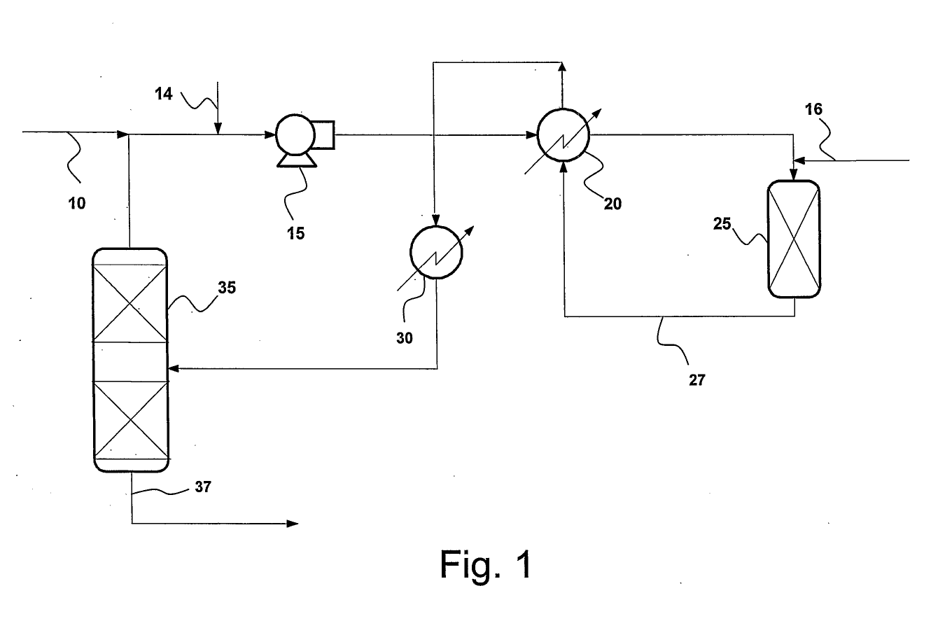

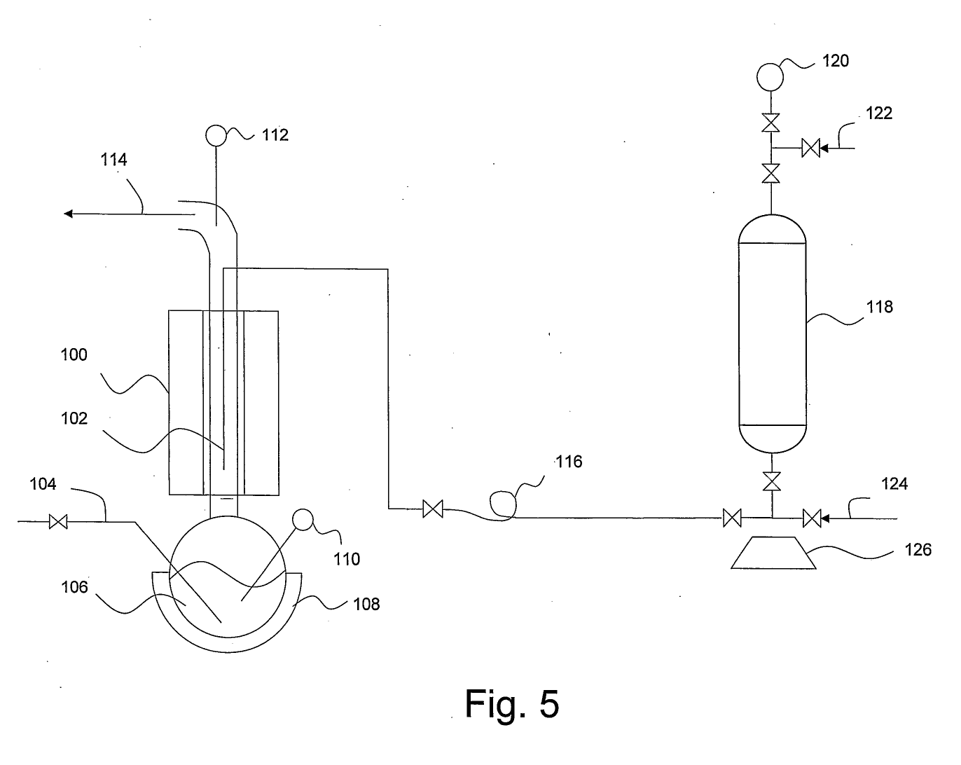

Unless otherwise specified, like numerals represent like parts among FIGS. 1-4 of the Drawings. In FIG. 5 the chief parts of the laboratory apparatus used to gather information and data, are identified by a different set of numerals.

### GLOSSARY

While every attempt is made herein to make things as clear as the English language will permit, a couple of terms are discussed here so as to avoid such misinterpretations as can presently be foreseen. Thus, the term “vanadium-containing catalyst” standing alone means a catalyst which may or may not be at least in part on a surface of a catalyst support and which catalyst in either such case (i) contains vanadium in one or more chemical forms which need not be metallic vanadium itself, (ii) when at one or more temperatures in the range of about 450 to about 700° C. can, in the presence of air, oxygen, or air enriched in oxygen, cause all or at least a portion of a quantity of sulfur dioxide in contact therewith to be converted (i.e., transformed, or changed into) to sulfur trioxide, and (iii) when in the form of a bed thereof having at least a portion of such bed at one or more temperatures in the range of about 450 to about 700° C. and through at least a portion of such bed there exists a gaseous flow comprised of sulfur dioxide, will allow release from the bed of a product gaseous stream comprised of sulfur trioxide.

Hereinafter there is reference made to a description of certain catalysts in a product bulletin of Monsanto Enviro-Chem which are useful in the practice of the present invention. In such brochure it is indicated that their catalysts are properly referred to as “vanadium-containing catalysts” since they are not vanadium pentoxide catalysts. Vanadium pentoxide catalysts are also useful in the practice of the present invention. Therefore, as the immediately preceding paragraph is believed to make manifestly clear, as used herein the term “vanadium-containing catalysts” standing alone is not to be construed as limited to only the materials referred to in the product bulletin of Monsanto Enviro-Chem. Rather the term as used herein, and unless otherwise specified, is used as a generic term which is not only inclusive of the materials referred to in the product bulletin of Monsanto Enviro-Chem but in addition to other vanadium-containing catalysts that will work in the manner described herein under the conditions and with the materials described herein, such as, for example, a supported or unsupported vanadium pentoxide catalyst.

The term “vaporized” with reference to sulfur such as in terms such as “sulfur is vaporized” or “the vaporized sulfur” or the like does not mean that the vapors must be composed exclusively of elemental sulfur in vaporized form. Rather the vapors are composed of whatever is produced when molten sulfur approaches and/or contacts the hot surfaces of the vanadium-containing catalyst bed which is at one or more temperatures in the range of about 450 to about 700° C. in the presence of a gaseous flow comprised of sulfur dioxide, sulfur trioxide, and air, oxygen, or air enriched in oxygen.

The term “vanadium-containing catalyst that oxidizes sulfur dioxide to sulfur trioxide” does not mean that in the practice of this invention, the catalyst only serves the function of oxidizing sulfur dioxide to sulfur trioxide. Because of the complexity of the gaseous mixtures in contact with the catalyst, other reactions referred to in the body of this document may occur. Thus, the qualification that the catalyst “oxidizes sulfur dioxide to sulfur trioxide” is a descriptor to identify one function that the catalyst must be able to perform.

The term “total moles of oxygen per total moles of sulfur” (in phrases such as “total moles of oxygen per total moles of sulfur present in the gaseous stream to which the sulfur is added” and “total moles of oxygen per total moles of sulfur present in the gaseous stream prior to the addition thereto of molten sulfur”) refers to the molar ratio of (a) the total moles of elemental oxygen, oxygen in sulfur dioxide, and oxygen in sulfur trioxide to (b) the total moles of elemental sulfur, sulfur in sulfur dioxide, and sulfur in sulfur trioxide, wherein the components of (a) and of (b) are those present in the system being referred to.

### FURTHER DETAILED DESCRIPTION INCLUDING FURTHER PREFERRED EMBODIMENTS

First of all, the reactor in which the fixed bed of vanadium-containing catalyst is disposed (positioned), can be in any position relative to ground level. A few non-limiting positions include for example horizontal, substantially horizontal, vertical, substantially vertical, upwardly inclined, downwardly inclined, and so on. In preferred embodiments the reactor is in an upstanding (upright) position. Also the reactor can have any shape and cross-sectional configuration that serves the purpose of enabling the process of this invention to be conducted therewith as described herein. Naturally, the reactor will need a gas inlet portion and a gas outlet portion and should be configured such that all or substantially all of the incoming gaseous stream will pass through the fixed bed catalyst that is maintained therein. Also, the reactor needs to be equipped with heating apparatus that will enable the catalyst to be heated (e.g., to one or more temperatures in the range of about 450 to about 700° C., and preferably in the range of about 450 to about 600° C.) during startup in order to cause the process to be initiated. Once initiated, the process is sufficiently exothermic as not to require addition of further heat during the course of the reaction as temperature control can be maintained by adjusting the feed rates to the reactor. Although the catalyst bed is kept at one or more temperatures in the range of about 450 to about 700° C., brief excursions outside of this range can usually br tolerated if the period of the excursion is sufficiently brief. In this connection, there can be zones in the catalyst bed at different temperatures within this range. In other words, the catalyst bed need not be at one and only one temperature throughout.

Since the reactor is continuously exposed to internal high temperature conditions during operation and since corrosive gases are being handled and produced within the reactor, it should be fabricated from suitable corrosive resistant materials. Alonized stainless steel reactors and reactors constructed of high nickel-content alloys serve as non-limiting examples of reactors made with suitable materials of construction. Two or more reactors may be used in tandem, if desired. Indeed, it is feasible to have multiple catalyst beds arranged in series with sulfur and oxygen or air feeds between each of them in order to moderate the exothermic nature of the oxidation reaction.

Various types of vanadium-containing catalysts can be used in the practice of this invention provided that the catalyst has the ability to oxidize sulfur dioxide to sulfur trioxide. For example, in addition to vanadium pentoxide, modified vanadium pentoxide catalysts such as described in U.S. Pat. Nos. 3,793,230 and 4,285,927 may be used. Also, a vanadium pentoxide catalyst can be on a suitable support so that structural integrity is maintained and so that the catalyst can otherwise withstand the high temperature(s) at which the bed is operated. Non-limiting examples of such supports include high temperature resistant ceramics, alumina, silica, silica alumina, zeolites, and similar materials.

Among preferred vanadium-containing catalysts used in the practice of this invention, are sulfuric acid catalysts such as are available from Monsanto Enviro-Chem as LP-120, LP-110, LP-220, T-210, T-516, T-11, Cs-120, Cs- 110, Cs-210, and presumably LP-1150. According to a product brochure by Monsanto Enviro-Chem concerning such sulfuric acid catalysts and obtained from their website on Apr. 13, 2004, the LP-120, LP-110, LP-220, Cs-120, and Cs-110 are available in the shape of rings, whereas T-210, T-516, T-11, and Cs-220 are available in the shape of pellets. Dimensions of the rings and pellets are given in that brochure. Reference to LP-1150 is not made in this brochure. According to this brochure, the main components of these catalysts include SiO₂ (silica as a support), vanadium (V), potassium (K), and/or cesium (Cs), and various other additives. It appears from this brochure that these catalysts may be formed from a molten salt mixture of potassium/cesium sulfates and vanadium sulfates, coated on a solid silica support. Monsanto Enviro-Chem further states that because of the unique chemistry of this molten salt system, vanadium is present as a complex sulfated salt mixture and “NOT” as vanadium pentoxide (V₂O₅). The brochure further states that the catalyst is more correctly called a “vanadium-containing” catalyst rather than the commonly-used “vanadium pentoxide” catalyst. It further appears from these brochures that LP-120, T-210, LP-110, and T-11 catalysts are potassium promoted, whereas Cs-120, Cs-110, and Cs-210 are cesium promoted. The cesium promoted catalysts are indicated to be more expensive, but capable of operation in a catalyst bed at lower temperatures in the range of 390-410° C. According to the material safety data sheets of Monsanto Enviro-Chem pertaining to the sulfuric acid catalysts T-516, T-210, T-11, LP-120, LP-110, LP-220, and LP-1150 have the chemical name “mixture of complex inorganic salts (oxosulfato vanadates) containing sodium, potassium, and vanadium salts on crystalline silica support. The composition of these materials is indicated to be 39-45 wt % of vanadium salt complex (no CAS No. available), 24-32 wt % of crystalline silica (CAS No.14464-46-1), and 26-28 wt % of amorphous silica (CAS No. 68855-54-9). There is also an indication the crystalline silica may comprise cristobalite and quartz. Typical physical data for these catalysts as given in the MSDS are as follows:

Appearance:

Yellow to light green pellets
7/32″ or 5/16″ in diameter by ⅜″ average length (Type 16, Type 210, Type 11)
½″ or ⅜″ in diameter by ½″ average length (Type LP-120, Type LP-110, Type LP-220)
½″ diameter by ⅝″ long Raschig Ring (Type LP-1150)
   Solubility:
65-75% SiO₂—insoluble
25-35% inorganic salts—partially soluble in water
   Bulk Density:
1.15 lb/L (Type LP-220)
   Specific Gravity bulk (H₂O=1)
0.60-0.70 (Types 516, 210, 11)
0.50-0.55 (Types LP-120, LP-110, LP-1150).

The residence time of the gases within the catalyst bed should be sufficient to enable high conversions to sulfur trioxide, and thus limited residence times (up to 5-10 seconds) are generally sufficient.

One of the preferred embodiments of this invention is to utilize a fixed bed of a vanadium-containing catalyst to oxidize sulfur dioxide into sulfur trioxide in an upstanding reactor (upright, in other words the reactor need not be perfectly vertical as it can lean or be tilted somewhat) with the incoming gaseous stream comprised of sulfur dioxide and oxygen and/or air (which stream preferably also contains sulfur trioxide) entering into the upper portion of the reactor into a headspace above the catalyst bed, and to introduce the molten sulfur above the upper end portion of the catalyst, preferably above or in an upper end portion of the headspace. In this way, the molten sulfur travels substantially downwardly toward the upper end portion of the catalyst bed and at least a portion if not substantially all of the sulfur is vaporized as it contacts and/or comes into close contact with the hot upper end portion of the catalyst. The provision of the headspace above the catalyst bed provides a zone in which at least some of the vapors produced by the vaporization of the sulfur and at least some of the incoming gaseous stream can come into contact with each other and be carried by the force of the incoming gaseous stream into the catalyst bed. Without desiring to be bound by theory, one may speculate that some oxidation of sulfur vapors may even be initiated in the lower regions of the headspace. But whatever takes place in the headspace, the end result is the release or emergence from a lower end portion of the catalyst bed of a gaseous stream enriched in sulfur trioxide with the catalyst bed itself remaining free of significant buildup or deposition of sulfur coatings on the catalytic surfaces.

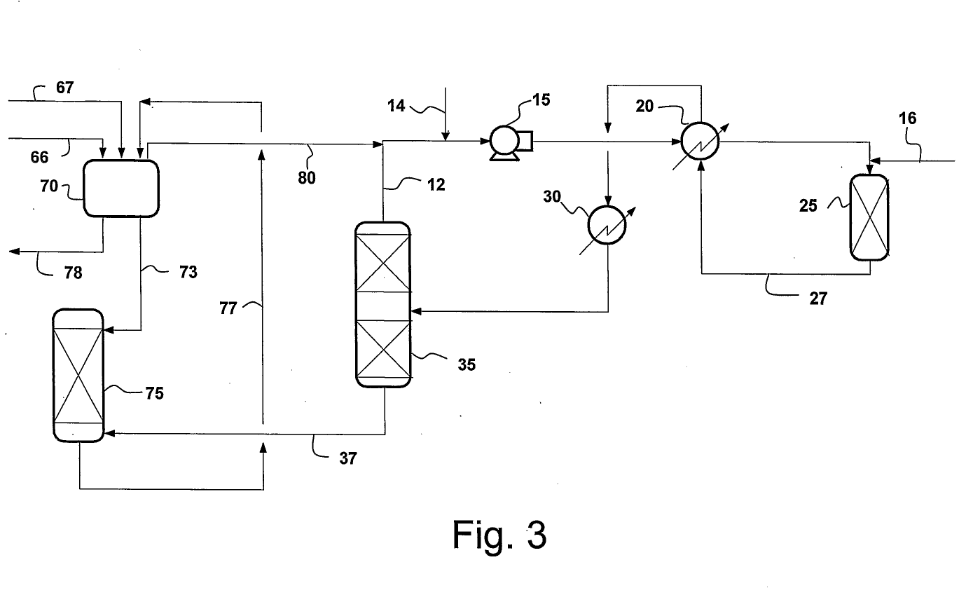

In preferred embodiments of this invention wherein the incoming gaseous stream approaching the location at which the molten sulfur is fed into the gaseous stream is comprised of sulfur dioxide, sulfur trioxide and oxygen and/or air, the amount of sulfur trioxide released or emerging from the downstream end portion of the catalyst bed is higher than the amount of sulfur trioxide that would be released or that would emerge from the same downstream end portion of the same catalyst bed under the same operating conditions and with the same incoming gaseous stream in the absence of the sulfur addition. Such increased amount of sulfur trioxide released or emerging from the downstream end portion of the catalyst bed is apparently due to the occurrence of at least two reactions in the process when the incoming gaseous feed stream contains sulfur trioxide as well as sulfur dioxide and air and/or oxygen. More specifically, in the absence of sulfur addition, each mole of SO₃ being fed through the oxidation catalyst is unchanged and provides one mole of SO₃ in the product released or emanating from the downstream end portion of the catalyst. However, with the addition of sulfur to this incoming feed stream pursuant to this invention, theoretically each mole of SO₃ upstream from the catalyst is converted in a two-step reaction sequence into 1.5 moles of SO₃ in the product emanating from the catalyst. These two consecutive reactions can be expressed as follows:
2SO₃+S→3SO₂ (1)
3SO₂+1.5O₂→3SO₃ (2)
Thus in addition to the conversion of the original SO₂ to SO₃, there is a theoretical 50% increase in SO₃ formation from the sequential reactions of equations (1) and (2) above, i.e., 2 moles of SO₃ becomes 3 moles of SO₃. Also, both of the reactions of equations (1) and (2) can be performed in association with the catalytic oxidation of the initial SO₂ in the gaseous stream to SO₃—which also proceeds as in Equation (2)—without need for expensive ancillary reaction equipment. In other words, the reaction of equation (1) which can be considered as oxidation of sulfur to SO₂ apparently takes place first at least to some extent, and then both the original SO₂ and the newly-formed SO₂ are catalytically oxidized via equation (2) to form SO₃ whereby an increase in total SO₃ formation occurs as compared to the same operation with the same quantities of materials except that no sulfur is fed. In fact, it is preferred to conduct these reactions in a simple reactor such as schematically depicted in FIG. 3 of the drawings. Thus in further preferred embodiments of this invention both the oxidation of sulfur into SO₂ and subsequent catalytic oxidation of SO₂ into SO₃ can occur at least in part in a single two-stage reactor or reaction zone in which there is a headspace above or a “dead” space ahead of the catalyst bed. Thus the preferred embodiments of this invention described in this paragraph involve a dichotomy in that while it is desired to produce sulfur trioxide from sulfur dioxide, the process of such preferred embodiments first actually appears to reduce the amount of sulfur trioxide originally present in the incoming gaseous stream by converting that sulfur trioxide to sulfur dioxide followed by the oxidation of at least a portion of that newly-formed sulfur dioxide into sulfur trioxide. Moreover, pursuant to this invention, in addition to enabling the production of greater amounts of sulfur trioxide by virtue of the feed of sulfur upstream from, or preferably just prior to, the oxidation of sulfur dioxide to sulfur trioxide, such processing does not result in any significant poisoning of the vanadium-containing catalyst, nor does the processing result in reduction of process or economic efficiency. Instead, the cost-effectiveness of the overall process can be significantly improved.

The same considerations are applicable in the embodiments of this invention wherein an improvement is provided in a process in which a first gaseous stream comprised of sulfur dioxide, sulfur trioxide and oxygen and/or air is passed through and in contact with a bed of a vanadium-containing sulfuric acid catalyst such as a vanadium-containing catalyst such as vanadium pentoxide, and preferably a bed of a mixture of complex inorganic salts (oxosulfato vanadates) containing sodium, potassium and vanadium salts on crystalline silica support, or a catalyst including silica as a support within a salt mixture comprised of potassium and/or cesium sulfates, and vanadium sulfates coated on the solid silica support, that oxidizes sulfur dioxide to sulfur trioxide and that releases therefrom a product gaseous stream comprised of sulfur trioxide. In these embodiments the improvement comprises oxidizing sulfur with air, oxygen and/or sulfur trioxide (preferably with a gaseous stream which contains (i) at least sulfur trioxide and air or oxygen, or (ii) sulfur trioxide, air and added oxygen) to form a second gaseous stream enriched in sulfur dioxide and introducing at least a portion of the second gaseous stream into the first gaseous stream to form a mixed gaseous stream, and passing the mixed gaseous stream into an upstream portion of the above catalyst bed maintained at one or more temperatures in the range of about 450 to about 700° C., and preferably in the range of about 450 to about 600° C. This results in the formation a product stream emanating from a downstream portion of the catalyst bed that is enriched in sulfur trioxide. The amount of sulfur trioxide in the product stream tends to be greater than could be predicted from the oxidation of the total amount of sulfur dioxide in the mixed gaseous stream to sulfur trioxide. The oxidation of sulfur in this embodiment of the invention is usually carried out in a separate reactor.

Referring to the preferred embodiment depicted in FIG. 1, a gaseous SO₂ feed at 10, which optionally contains some SO₃, and a recycled gaseous stream containing oxygen and/or oxygen-depleted air (mainly nitrogen) and, optionally (but preferably), a minor amount of SO₃ at 12, are mixed with fresh air or oxygen from 14 and the resultant gaseous mixture is drawn into blower 15. Blower 15 propels the resultant mixture through indirect heat exchanger 20 which in part heats this gaseous mixture. Into the heated gaseous mixture is then injected molten sulfur from 16 whereby when SO₃ is present in the recycled gaseous stream at 12, additional SO₂ is deemed to be formed by reaction of sulfur with SO₃. The resultant enriched SO₂ stream is then passed into reactor 25 containing a suitable vanadium-containing catalyst (most preferably a fixed bed of supported vanadium-containing catalyst) for oxidizing SO₂ to SO₃. This gaseous product mixture enriched in SO₃ is passed from reactor 25 as at 27 and through heat exchanger 20 wherein heat from the exothermic reaction in reactor 25 is employed to heat the mixture coming from blower 15, and thereby reduce the temperature of the gaseous mixture coming from reactor 25. This latter mixture is then further cooled in cooler 30 and then passed into distillation column 35. The distillation column is operated so as to remove the more volatile components of the mixture as the overhead which thus constitutes the recycled gaseous stream referred to at the outset. The desired SO₃ is taken from the bottom of column 35 as at 37.

The makeup of the recycled gaseous stream will vary somewhat depending on whether air, oxygen, or air enriched with oxygen is fed to the gaseous mixture upstream from the place where the sulfur from 16 is introduced. If pure oxygen is fed, the recycled gaseous stream will contain unreacted gases and in preferred embodiments, will also contain some SO₃. If air or air enriched with oxygen is used, the recycled gaseous stream will contain nitrogen as well as other unreacted gases and in preferred embodiments, will also contain some SO₃. In either case where SO₃ is present, the proportion of SO₃ in the recycled gaseous stream typically will be less than about 10 percent by volume.

In the gaseous stream heading toward the place where the sulfur from 16 is introduced, the ratios of SO₂ to SO₃ can also vary. Typically this ratio on a molar basis will be in the range of about 15:1 to about 25:1, and preferably in the range of about 22:1 to about 24:1. The amount of sulfur fed from 16 should be at least about 0.002 mole per mole of SO₂ in the gaseous mixture to which the sulfur is added, and typically will be in the range of approximately 0.005 to 0.020 mole, and preferably in the range of approximately 0.015 to 0.018 mole, of sulfur per mole of SO₂ in such gaseous mixture. Greater amounts of sulfur can be used, but ordinarily will serve no useful purpose. Also with one or more reactors having an operating temperature limit of about 1200° F. (ca. 649° C.) there should be at most about 2.0 moles of molecular oxygen (O₂) per mole of sulfur as SO₂ and elemental sulfur present in the gaseous stream entering the catalyst bed. Typically with reactors having such operating temperature limit, this gaseous stream will contain in the range of approximately 1.25 to 1.75 moles of molecular oxygen per mole of sulfur as SO₂ and elemental sulfur present in such stream. With reactors having higher operating temperature limits, more than about 2.0 moles of molecular oxygen per mole of sulfur as SO₂ and elemental sulfur can be present in the gaseous stream entering the catalyst bed. The flow rates of oxygen, SO₂-SO₃ gaseous mixture (or SO₂ when an SO₂-SO₃ gaseous mixture is employed), and sulfur are measured and monitored using flowmeters. The amount of SO₃ is calculated based upon the distillation temperature relative to the mole fraction SO₃ at a specific distillate temperature.

Molten sulfur can be introduced to the gaseous mixture in different ways. For example the gaseous mixture can be passed under pressure through the molten sulfur or the molten sulfur can be sprayed into the gaseous mixture. Preferably the molten sulfur is initially conveyed by pumping the molten sulfur into downwardly flowing gaseous stream approaching the reactor so that both the force of the stream and gravity cause the sulfur to proceed downwardly toward and/or onto the upper end portion of the catalyst bed where the sulfur is vaporized. However introduced, the molten sulfur will almost instantaneously react with SO₃ in the incoming stream to form, in situ, additional SO₂, and thus at that point reduce or eliminate the SO₃ content in the stream.

In plant installations involving exothermic reactions, the chemical processing equipment is typically operated below a projected thermal maximum which includes a selected margin below the maximum rating for the equipment being used. It follows therefore that in order to operate safely, operational limitations are typically equipment-related thermal limitations due to the exotherms of these oxidations. For example, a given plant for use in the practice of this invention may be thermally rated for operation at 650° C. (1200° F.) or 750° C. (1382° F.).

Thus the temperatures used in the process can vary depending upon the particular equipment used and the rates and volumes at which the materials are being processed provided of course that the sulfur fed to the gaseous stream is fully vaporized. Generally speaking, the temperatures of the zone in which the molten sulfur reacts with the SO₃ in the incoming stream are typically within the range of about 232 to about 600° C. and preferably in the range of about 450 to about 600° C. for incoming gaseous feeds in the range of about 2500 to about 10,000 kg/hour, provided the equipment can be safely operated under these conditions. As noted above, the temperatures in the oxidation catalyst zone are typically in the range of about 450 to about 700° C. and preferably are in the range of about 450 to about 600° C. When a supported or unsupported vanadium-containing catalyst is used, the temperature(s) in the catalyst bed should not reach the temperature at which the unsupported catalyst melts or loses its ability to perform its role as a catalyst or at which the supported catalyst melts, loses its ability to perform its role as a catalyst, undergoes material degradation or suffers loss of structural integrity. Departures from the foregoing ranges are permissible and are within the scope of this invention provided that the departures do not materially interfere with the process and do not constitute hazardous operating conditions in relation to the processing equipment being used.

Operating pressures of at least 10 psig, and preferably at least 100 psig are preferred for converting SO₂ to SO₃. Maximum pressures are preferably about 150 psig, but can be higher if desired.

As noted above, it is particularly preferred to conduct both the reduction of SO₃ by sulfur to form SO₂ (which is also oxidation of sulfur to SO₂) and the subsequent catalytic oxidation of SO₂ into SO₃ in reactor 25. In such case, reactor 25 comprises mixing sections in which vaporized sulfur reacts with SO₃ to enrich, in situ, the SO₂ concentration, and a catalytic section in which the oxidation of SO₂ to SO₃ takes place.

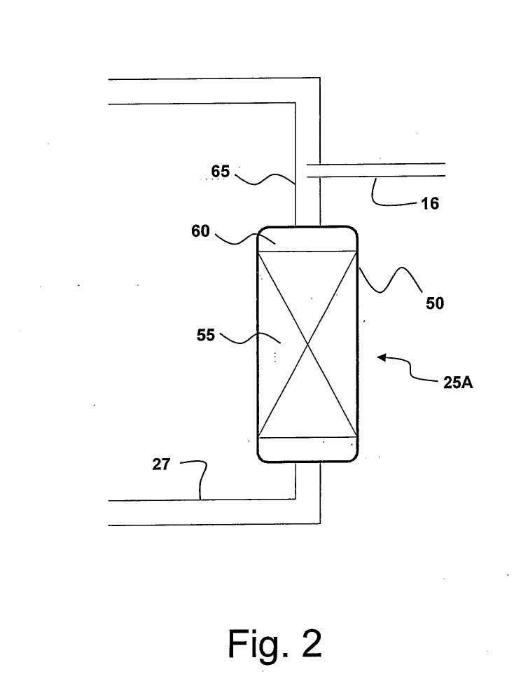

Although various configurations for reactor 25 can be used, FIG. 2 schematically depicts a reactor generally referred to as 25A as a preferred configuration for such reactor 25. Reactor 25A itself comprises housing 50 containing a fixed catalyst bed 55 having a headspace or dead volume 60 (an unoccupied zone) above the top of the bed. During operation, at least a portion of bed 55 is maintained at one or more suitable operating temperatures in the range of about 450 to about 700° C. and preferably in the range of about 450 to about 600° C. so that oxidations such as result in the formation of additional sulfur trioxide take place therein. Leading to dead volume 60 is conduit 65 into which is forced molten sulfur from 16 so that the molten sulfur is transported downwardly both by gas flow and gravity through an entry port in the top of reactor 25A and all or at least a portion thereof impinges upon the top of the hot catalyst bed. Note that some or possibly most if not all of the sulfur may possibly vaporize before reaching the catalyst bed. On approaching and/or impinging upon the catalyst bed the molten sulfur is vaporized and the sulfur vapors mix with the incoming gases in headspace or dead volume 60 and are swept by the incoming gases into the catalyst bed where all or substantially all SO₂ is oxidized to SO₃ utilizing oxygen from air and/or oxygen fed upstream as at 14. A gaseous product stream enriched in SO₃ is taken from reactor 25A via conduit 27.

In a typical plant installation employing a supported vanadium-containing catalyst and incoming gaseous stream containing SO₂ and a small amount of SO₃, the approximate heat-limited flowrates around reactor 25 are 20,000 lb/hr (9072 kg/hr) of SO₂/SO₃ gaseous mixture containing in the range of about 0.010 to about 0.042 mole of SO₃ per mole of SO₂, 175 lb/hr (79 kg/hr) of sulfur, and 560 lb/hr (254 kg/hr) of oxygen. In an operation of this scale, reactor 25A having a total height of about 10 feet, and a diameter of 3 feet with a 4-foot high headspace or dead volume 60 and a 6-foot high catalyst bed packed with supported vanadium-containing catalyst having an average particle size of about 0.25-0.50 inch, has been found very suitable. Reactor 25A is preferably constructed of alonized stainless steel.

Initially the reactor is heated by an indirect gas-fired furnace and the temperature is easily maintained from the resulting exotherms of the oxidation reactions.

Distillation column 35 is used to separate the recycle mixture of SO₂/SO₃ and inerts from the product SO₃. As with the reactor, it is preferred to run the column under 100 psig although higher pressures can be used, within normal high-pressure equipment limitations.

Conversions of SO₂ to SO₃ of at least about 99.8% are achievable by proper conduct of this invention.

It will be readily apparent that the flow diagram of FIG. 1 can be modified without departing from the scope of this invention. For example, the incoming gaseous stream approaching the sulfur feed at 16 can be devoid of SO₃. Similarly, whether or not SO₃ is in such gaseous stream, the oxygen and/or air feed from 14 can occur at other locations such as downstream from the blower 15, or to the gaseous effluent from distillation column 35. It is desirable however that the addition of the oxygen and/or air occur upstream from the feed of the molten sulfur. Also, higher ratios of SO₃:SO₂ are possible in the inlet of the reactor. Molten sulfur can be added to a horizontally disposed reactor 25 provided the sulfur is carried by the gaseous stream into the hot catalyst bed or that the sulfur drops onto the front portion of the hot catalyst bed and is vaporized thereby and swept into the rest of the catalyst bed. Another variant of the invention is to cause the molten sulfur to impinge upon a hot high temperature resistant inert ceramic or inert metal surface disposed (positioned) at a suitable location ahead of the catalyst bed so that the sulfur is vaporized in the incoming gaseous stream and is carried into the catalyst bed.

In FIG. 3, there is schematically depicted therein a process flow diagram of a preferred embodiment of this invention, namely utilization of the above-described SO₃ generation process for use in the production of another product such as a brominated flame retardant. Without external isolation, the SO₃ eluent from the distillation is directly used to achieve electrophilic aromatic bromination using a highly deactivated substrate (reactant A) of structure which prohibits toward the use of common Lewis acid catalysts, such as AlCl₃, AlBr₃, FeCl₃, FeBr₃, etc., for this reaction. Those strongly deactivated aromatic substrates (having substituents with Hammett σₚ>0.2) and specifically those which, by virtue of their substructures would not be amenable to use of these catalysts, are included in the scope of this invention. This rather unique reaction medium facilitates production of commercially important flame retardants, such as tetrabromophthalic anhydride, by reaction with a halogenating agent (reactant B), such as bromine. See in this connection U.S. Pat. Nos. 3,382,254 and 5,288,879.

Referring more specifically to FIG. 3, reactants A (e.g., phthalic anhydride) and B (e.g., bromine) are cofed via lines 66 and 67 respectively into the reactor 70 along with an activating solvent, namely a concentrated oleum. This concentrated oleum, typically 25-65% SO₃ by weight, is generated by passing SO₃ formed as described above, for example in connection with FIG. 1, and taken from the bottom of column 35 via line 37 into tower 75. In tower 75 the SO₃ is mixed with depleted oleum (e.g., 22% oleum) coming from reactor 70 via line 73 so that in tower 75 a more concentrated oleum (e.g., 65% oleum) is regenerated and delivered from the bottom of tower 75 into reactor 70 as indicated via line 77. Any SO₂ and SO₃ emanating from reactor 70 are recycled as indicated via line 80 to the system such as schematically depicted in both FIGS. 1 and 3 (except that the stream in line 80 in FIG. 3 corresponds to the feed in line 10 in FIG. 1), wherein the SO₂ (and SO₃ if present) are converted into an enriched stream of SO₃ taken from the bottom of column 35, which stream is used in regenerating the oleum in tower 75. Upon completion of the electrophylic aromatic bromination, the solid product is recovered as at 78 and processed downstream therefrom for packaging.

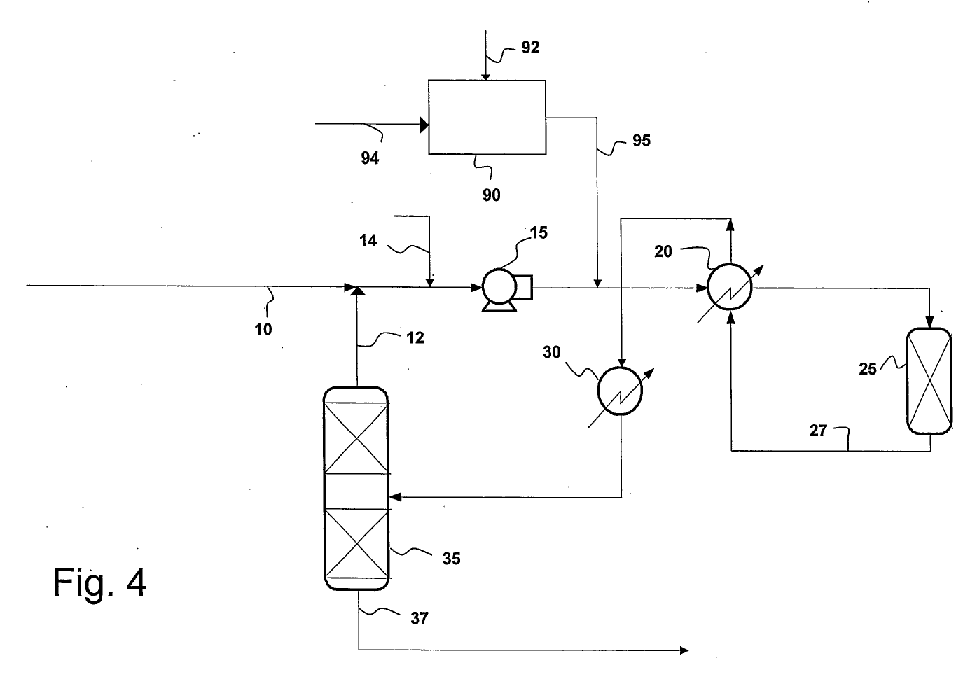

The schematic flow diagram of FIG. 4 depicts another way pursuant to this invention of carrying out a process of producing SO₃ from a gaseous stream of SO₂ containing oxygen and/or air and a minor amount of SO₃. It will be seen that FIG. 4 is the same as FIG. 1 as described above except that reactor 90 is provided, and that the molten sulfur feed at 16 of FIG. 1 is omitted (but could be included if desired). Fed into reactor 90 are molten sulfur as at 92, and an oxidant as at 94. The oxidant can be sulfur trioxide or air and/or oxygen, or it can be any combination of these. The oxidant preferably is or includes sulfur trioxide. When two or all three of air, oxygen, and sulfur trioxide are used as the oxidants they can be fed separately or as one or more preformed mixtures of gaseous oxidants. Alternatively, the sulfur is melted after being charged and then one or more feeds of the gaseous oxidant(s) employed are initiated, such that a gaseous stream enriched in sulfur dioxide is propelled out of reactor 90 and into the gaseous stream as at 95 to form a mixed stream which is carried into the catalyst bed in reactor 25. It will be appreciated that the oxidations in reactor 90 are uncatalyzed oxidation reactions. When sufficient air or oxygen to conduct the oxidation(s) occurring in reactor 25 is introduced by the stream as at 95, the feed at 14 may be omitted, if desired. Otherwise the operation depicted in FIG. 4 is as described above in connection with FIG. 1. Note that the gaseous feed as at 95 can be at any suitable location upstream from reactor 25.

The following Examples demonstrate the efficacy of the process of this invention in producing high yields of sulfur trioxide from gaseous streams containing both SO₂ and some SO₃. In particular, these Examples involve tests demonstrating that the temperature depend equilibrium which liberates SO₃ (from thermally cracking H₂SO₄) can be used along with sulfur to generate additional SO₂. These Examples are presented for purposes of illustration and not limitation with respect to the generic scope of this invention.

### EXAMPLE 1

A 1-inch by 24-inch quartz furnace tube was filled with ¼-inch ceramic Berl saddles containing 2.02 g of sulfur (preloaded). The materials were placed inside a furnace operated at 450° C. and the exit vent was fitted with a trap containing 18 g of NaOH as an 11.7 wt % aqueous solution in a gas absorption bottle. Sulfuric acid (106.35 g) was pumped into the furnace tube during a period of about 2.5 hours with observation of steam reflux which included traces of vaporized sulfur. The exit gas was trapped as sodium sulfite (Na₂SO₃) and analyzed using excess iodine and then back-titrated with sodium thiosulfate. The SO₂ yield was 49.33%.

### EXAMPLE 2

The procedure of Example 1 was repeated using 2.16 g of sulfur (preloaded) and 24.72 g of 96% H₂SO₄, which were placed inside the furnace (operated at 450° C.) and the exit vent was fitted with a trap comprised of a 250 mL gas absorption bottle containing 152.05 g of 21.1 wt % aqueous NaOH. Sulfuric acid was pumped into the furnace tube for a period of about 0.3 hour with observation of steam reflux which included traces of vaporized sulfur. The exit gas was trapped with sodium sulfite (Na₂SO₃) and analyzed using excess iodine and then back-titrated with sodium thiosulfate. The SO₂ yield was 50.8%.

### EXAMPLE 3

The procedure of Example 1 was repeated except that 4.5 g, 140 mmols of sulfur were preloaded into the furnace tube and reacted with 81 mL (149.04 g, 1.52 mols) of H₂SO₄ in a 1-inch by 18-inch pipe heated to 452-464° C. inside a furnace. The exit gas was trapped using aqueous NaOH and analyzed as in Example 1. The total SO₂ yield was 86%.

### EXAMPLE 4

A study was carried out in order to determine whether sulfur could be oxidized by SO₃ at temperatures in the range of about 700-1100° F. (ca. 370-593° C.) and whether the other compounds in the sulfur would also be oxidized or form solids and plug a vanadium oxide catalyst bed in the reactor. Accordingly, a continuous, laboratory scale, sulfur oxidation unit was constructed. This unit is schematically depicted in FIG. 5.

In FIG. 5 the following numerals represent the following parts 100 is a tube furnace, 102 is an SO₃ heat tube, line 104 is a nitrogen feed, 106 is molten sulfur, 108 is a heating mantle, 110 is a sulfur temperature gauge, 112 is an overhead temperature gauge, and 114 is a vent line to an empty flask and scrubbers, 116 is telfon tubing, 118 is a sulfur trioxide feed tank, 120 is a feed tank pressure gauge, 122 is a nitrogen feed for tank pressurization, 124 is a nitrogen feed for post experiment flushing of the system, and 126 is an SO₃ balance. It will be noted from FIG. 5 that nitrogen at a flowrate of 1.0 standard liter per minute (SLPM) was used to vaporize the sulfur into the reaction tube, which is heated in a tube furnace. The sulfur trioxide was fed from a stainless steel feed tank as a liquid and preheated in its feed tube prior to entering the reaction zone. The surface temperature of the SO₃ feed tank was maintained at 70-80° C. to ensure the SO₃ was liquid. The furnace temperature was maintained at 700° F. (ca. 370° C.) throughout each of four experimental runs. The first two of these runs involved use of excess sulfur whereas the third and fourth runs involve use of excess SO₃.

The exit gases were passed through an empty flask to knock-out any entrained liquids and then were collected in a series of two scrubbers. In the first run, the first scrubber contained water (800 gms) and the second scrubber contained a 25 wt % caustic solution (840 gms). The caustic scrubber was maintained at 40° C. to prevent Na₂SO₃ precipitation and pluggage of the dip-tube. Accurate analysis of the scrubber contents for SO₂ and SO₃ was difficult with this approach. After the first experimental run, the materials in these scrubbers were changed to improve the analysis. Following this change, the first scrubber contained a bromine/water mixture (25 Ogms/380.2 gms) while the second scrubber contained only water (800 gms). The first scrubber oxidized the SO₂ to SO₃ by the following reaction:
SO₂+2H₂O+Br₂→H₂SO₄+2HBr
The second scrubber trapped the SO₃ and HBr exiting from the first scrubber. Samples of each scrubber were collected and analyzed for wt % bromide and wt % acid. During each experimental run, the unit was operated for 35-60 minutes. Conditions spanning a wide range of sulfur:SO₃ molar ratios (2.4:1 to 0.25:1) were investigated.

In order to address the concern regarding the buildup of solids and potential pluggage of the vanadium-containing catalyst bed by the other components present in the sulfur, a sample of commercial plant sulfur was burned with air to determine the amount of material remaining. Sufficient material was burned to collect enough residue for analysis by ICP.

Table 1 contains a summary of the results from these four sulfur oxidation experimental runs. The amount of sulfur conveyed with nitrogen was calculated with ChemCad by assuming the nitrogen was saturated with sulfur vapors at the molten sulfur conditions. Note that the molten sulfur was held at 365-390° C. for the excess sulfur experiments (runs 1 and 2) and at 290° C. for the excess SO₃ experiments (runs 3 and 4). The SO₃ flowrate was varied from 1.0 to 1.8 gms/min. Even though equipment problems existed with all but one of the four experimental runs conducted, the results indicate that a substantial amount of the SO₃ and sulfur react to form SO₂ under suitable reaction conditions.

Throughout each experiment, the average reactor residence time was maintained at approximately 2-3 seconds. During periods where the residence time through the reactor was shorter, a blue liquid was observed to exit the reactor and collect in the knock-out flask. A sample was collected and analyzed by GC-MS. While both solution (with methylene chloride/methanol and acetonitrile) and headspace analysis revealed only SO₂, a residue was observed on the GC injection port liner. Literature references (Kagramanov, et al., “Physicochemical Properties of a Sulfur-Fuming Sulfuric Acid System”, Zh. Prikl. Khim., 1987, 60(10), pp 2177-82; Weast (ed.), CRC Handbook of Chemistry and Physics, 56th Ed., Boca Raton, Fla., 1975) suggest that this blue liquid is S₂O₃ an intermediate in the desired reaction.

TABLE 1
Summary of Oxidation Runs 1-4

| | | Limiting Reagent | | |
|---|---|---|---|---|
| Run | | (SO₃ or sulfur) to | Percent Closure | |
| No. | Conditions | SO₂ Conversion | on SO₃ | Comments |
| 1 | 380% | 67.8% | 90.9 | N₂ flow stopped during latter portion of |
| | excess sulfur | | | the run due to sulfur pluggage. Since SO₃ |
| | | | | feed was continued up to the point where |
| | | | | the plug was recognized, actual SO₃ |
| | | | | conversion is expected to be higher than |
| | | | | calculated. |
| 2 | 78% | 89.1% | 83.8 | — |
| | excess sulfur | | | |
| 3 | 103% | ca. 100% | 99.8 | N₂ (and, hence, sulfur) flow must have |
| | excess SO₃ | | | been slightly higher than 1 SLPM |
| | | | | because initial calculations indicated |
| | | | | that 142% of the sulfur was converted to |
| | | | | SO₂ based upon the SO₃ collected. |
| 4 | 66% | 89.7% | >100 | The number of problems experienced |
| | excess SO₃ | | | with the SO₃ feed caused sufficient |
| | | | | balance drift to cause >100% closure on |
| | | | | SO₃. |

### EXAMPLE 5

To determine the residue contained in the sulfur after oxidation, sulfur (267.5 gms) was added to a quartz tube and placed in a tube furnace. Air was fed continuously to the bottom of the tube through a quartz dip-tube. The furnace temperature was maintained at 380° C. Both oxidation and vaporization of the sulfur occurred. The vaporized sulfur was condensed and collected in a round-bottom flask. The SO₃ produced from oxidation was trapped in a large scrubber. The exit gas temperature was monitored throughout the oxidation process. Over the 6-8 hr period during this experimental run, the exit gas temperature fluctuated between 270 and 340° C. The majority of the residue was produced after the molten sulfur level in the tube fell below the dip-tube exit where the air was being fed. Prior to this point in this run, most of the oxidation occurred below the surface of the molten sulfur. Since operation temperatures were well above the sulfur auto-ignition temperature (232.2° C.) the liquid surface ignited after the sulfur liquid level fell below the dip-tube exit. All subsequent oxidation occurred at or above the gas-liquid interface. A rapid increase in the exit gas temperature to a maximum value of 473° C. was also observed after surface ignition. Oxidation during these conditions was less efficient and left more residue. The residue collected during this experiment was ash-like in form and was estimated at 84 ppm based upon the initial sulfur charged. A sample of this residue was collected end analyzed by ICP. Results of this analysis are provided in Table 2. Note that the residue is primarily comprised of sulfur and components contained in quartz. Sulfur samples were also submitted for a sulfur-ash test. The residue contents (after vaporization at 300° C.) for the starting material and the vaporized sulfur collected during the oxidation experiment were determined from the ash test to be 241 and 79 ppm, respectively. After ignition at 1000° C., the residue contents for these same samples were 28 and 54 ppm, respectively.

TABLE 2
ICP Results from Sulfur Residue Analysis

| Component | Result, ppm | Component | Result, ppm |
|---|---|---|---|
| Ag | <20 | Al | 840 |
| As | <20 | B | 130 |
| Ba | 650 | Be | <23 |
| Ca | 520 | Cd | <3 |
| Co | <3 | Cr | 370 |
| Cu | 1100 | Fe | 2560 |
| K | 270 | Li | 50 |
| Mg | 290 | Mn | 32 |
| Mo | 28 | Na | 1190 |
| Ni | 150 | P | 850 |
| Pb | <20 | S | 340,000 |
| Sb | <20 | Se | <20 |
| Si | 6940 | Sr | 9.1 |
| Ti | 33 | Tl | <10 |
| V | <3 | Zn | 390 |

### EXAMPLE 6

A separate sample of commercial plant sulfur was submitted to an outside laboratory (Galbraith Laboratories, Inc.) to determine the carbon content. The commercial plant sulfur was determined to contain 0.19±0.01 wt % carbon. In order to determine whether this carbon would be sufficiently oxidized by SO₃, amorphous carbon powder (5 gms) was placed in a furnace tube with approximately 3-4 inches of glass beads on top to prevent powder entrainment. Sulfur trioxide (approximately 80 gms) was fed over a 1.5-hour period through the carbon bed with the furnace temperature set at 538° C. (1000° F.). Following this feed period, the tube was allowed to cool and then was weighed to determine the carbon remaining. Approximately 1.8 gms of carbon was estimated to have been oxidized during the experiment. The remaining carbon was displaced to one side of the tube bottom which suggests that some SO₃ may have by-passed the carbon after a period of time. The results of this experiment indicate that SO₃ will oxidize the carbon that enters with the sulfur at the 700-1100° F. oxidation bed operating temperatures. A small amount of carbon powder escaped from the furnace tube and was trapped in the liquid SO₃ collected in the condenser attached at the top of the tube. While no carbon was observed to be exiting the condenser, the SO₃ puddles in the condenser became clearer after a period of approximately 10-20 minutes. From this observation, it was concluded that oxidation of the carbon could occur at lower temperatures as well.

The results obtained in Examples 4-6 indicate that sulfur trioxide (SO₃) will oxidize sulfur to form sulfur dioxide (SO₂) at suitable operating temperatures used in the oxidation of SO₂ to SO₃. Conversion of sulfur in a 2-3 second residence time gas phase in a plug-flow reactor at 700° F. (ca. 370° C.) should be at least 90% or greater. Small amounts of the reaction intermediate S₂O₃ was observed to exit the reactor at shorter residence times. Sufficient residue was collected from oxidation and vaporization of the commercial plant sulfur to perform an elemental analysis. This residue was primarily comprised of un-oxidized sulfur and elements contained in the quartz tube used during the test. The commercial plant sulfur was analyzed for carbon content and determined to be 0.19±0.01 wt %. The additional experiment of Example 6 suggests that carbon oxidation will occur at 700-1100° F. (ca. 370-593° C.) operating temperatures often used in the oxidation of SO₂ to SO₃. Carbon oxidation was observed to occur at lower temperatures as well.

### EXAMPLE 7

In an experimental plant scale operation using a system comparable to that of FIG. 1, sulfur was fed through a 1/16-inch nozzle in a 1-inch line at 145.9 lb/hr and reacted with oxygen and SO₃ (combined flowrate of incoming gases was 21206 lb/hr). The incoming gas composition to the oxidation reactor was approximately 92% SO₂, 5% SO₃, 2% oxygen with trace amounts of Br₂ and nitrogen. The outgoing gas composition from the oxidation reactor was approximately 87% SO₂ and 12% SO₃ with trace amounts of bromine, nitrogen, and oxygen. Incoming reactor gas temperature to the oxidation reactor was 850° F. (ca. 454° C.) and the outgoing gas temperature from the oxidation reactor was 1108° F. (ca. 598° C.). The SO₃ produced from this system was of sufficient reactivity for use in the operation described in relation to FIG. 3, an operation which had previously been conducted only with commercial oleum.

Brief Resume

From the foregoing description of this invention it will be seen that this invention in its broadest aspects involves providing a catalyst zone which can be in a single reactor or in two or more reactors in series. One or more vanadium-containing catalysts beds are in the reactor(s), preferably as one or more fixed beds. Different vanadium-containing catalysts can be used in the vanadium-containing catalyst bed(s). At least one bed, and preferably all of the beds, in the catalyst zone contain a vanadium-containing catalyst that oxidizes sulfur dioxide to sulfur trioxide. Fed into the bed (or a first bed in a series of beds) is a first gaseous stream comprised of sulfur dioxide, sulfur trioxide, and air or oxygen or air mixed with additional oxygen. (Air mixed with additional oxygen is also known as air enriched with oxygen). Emanating from the bed (or a last bed in a series of beds) is a second gaseous stream enriched in sulfur trioxide.

A feature of one such process is that sulfur, preferably molten sulfur, is introduced into at least the first gaseous stream so that the sulfur is vaporized and carried into the catalyst bed of the reactor (or a first catalyst bed in a series of catalyst beds) so that the stream emanating from the first catalyst bed (or from the first catalyst bed in a series of catalyst beds) is enriched in sulfur trioxide. When only one catalyst bed is used, the stream emanating therefrom is the above second gaseous stream. When more than one catalyst bed is used, the stream emanating from the last catalyst bed in the series is the above second gaseous stream. When more than one catalyst bed is used, sulfur can be fed upstream from each catalyst bed so that vaporized sulfur is formed from the sulfur of each sulfur feed and such vaporized sulfur enters its downstream catalyst bed. Preferably, additional sulfur trioxide, and/or air or oxygen or air mixed with additional oxygen, is also fed upstream from each catalyst bed. Additionally, when more than one catalyst bed is used, heat removal from the stream emanating from each successive bed should be carried out because of the highly exothermic reactions taking place in the reactors.

A feature of another such process is that sulfur dioxide is generated from sulfur and sulfur trioxide, and/or air, oxygen, or air mixed with additional oxygen and the resultant sulfur dioxide-containing gaseous stream is added to the first gaseous stream referred to above. In other respects, this process embodiment is generally similar to the system described in the immediately preceding paragraph except that sulfur itself is not introduced into the first gaseous stream referred to above.

In all of the processes referred to in this resume, the make up of the feed(s) to the catalyst bed is/are preferably adjusted or controlled (or the feeds to the catalyst beds are preferably adjusted or controlled), and the amount(s) of sulfur and any additional air or oxygen or air mixed with additional oxygen added to the stream heading to the catalyst bed is/are preferably adjusted or controlled (or the feeds to the catalyst beds are preferably adjusted or controlled) to the numerical values given hereinabove so that significant increases in sulfur trioxide content in the final product stream are achieved.

Compounds referred to by chemical name or formula anywhere in this document, whether referred to in the singular or plural, are identified as they exist prior to coming into contact with another substance referred to by chemical name or chemical type (e.g., another component, a solvent, or etc.). It matters not what chemical changes, if any, take place in the resulting mixture or solution, as such changes are the natural result of bringing the specified substances together under the conditions called for pursuant to this disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

All documents referred to herein are incorporated herein by reference in toto as if fully set forth in this document.

This invention is susceptible to considerable variation within the spirit and scope of the appended claims. Therefore the foregoing description is not intended to limit, and should not be construed as limiting, the invention to the particular exemplifications presented hereinabove. Rather, what is intended to be covered is as set forth in the ensuing claims and the equivalents thereof permitted as a matter of law.

1. In a process wherein a first gaseous stream comprised of sulfur dioxide, sulfur trioxide, and oxygen and/or air is passed into a bed of a vanadium-containing catalyst that oxidizes sulfur dioxide to sulfur trioxide and that releases therefrom a second gaseous stream comprised of sulfur trioxide, the improvement which comprises providing vaporized sulfur in said first gaseous stream so that the resultant mixture passes into the catalyst bed, and maintaining at least a portion of the catalyst bed at one or more temperatures in the range of about 450 to about 700° C. so that the second gaseous stream released from a downstream portion of said catalyst bed has an enriched content of sulfur trioxide.

2. (canceled)

3. The improvement as in claim 1 wherein at least a portion of said vaporized sulfur is formed by vaporizing sulfur within said first gaseous stream so that vaporized sulfur comes into contact with at least a portion of said first gaseous stream above and/or in an upstream portion of said catalyst bed.

4. (canceled)

5. The improvement as in claim 3 wherein the vaporized sulfur is formed by introducing molten sulfur into said first gaseous stream so that the sulfur is vaporized and the vaporized sulfur is mixed into and carried by said first gaseous stream into contact with an upstream portion of said catalyst bed.

6. (canceled)

7. The improvement as in claim 1 wherein said vanadium-containing catalyst is a fixed bed catalyst comprised of a vanadium pentoxide catalyst that oxidizes sulfur dioxide to sulfur trioxide and that releases therefrom a product gaseous stream comprised of sulfur trioxide.

8. The improvement as in claim 1 wherein said vanadium-containing catalyst is a fixed bed catalyst comprised of a mixture of complex inorganic salts containing sodium, potassium and vanadium salts on crystalline silica support, or a catalyst including silica as a support within a salt mixture comprised of potassium and/or cesium sulfates, and vanadium sulfates coated on the solid silica support, which fixed bed oxidizes sulfur dioxide to sulfur trioxide and that releases therefrom a product gaseous stream comprised of sulfur trioxide.

9. The improvement as in claims 3 or 5 wherein said one or more temperatures are such that said catalyst bed remains free of amounts of sulfur deposit formation and/or formation of sulfur coatings on said catalyst that would, if formed in such amounts, materially interfere with the catalytic activity of said catalyst.

10. A process of forming sulfur trioxide from sulfur dioxide, which process comprises:
a) passing a first gaseous stream comprised of sulfur dioxide, sulfur trioxide, and oxygen and/or air downwardly into a headspace in an upstanding reactor containing a fixed bed of a vanadium-containing catalyst that (i) oxidizes sulfur dioxide to sulfur trioxide, (ii) has an upper end portion and a lower portion below said upper end portion, and (iii) that releases therefrom at a lower portion thereof a second gaseous stream comprised of sulfur trioxide;
b) introducing into said first gaseous stream molten sulfur above the upper end portion of said fixed bed of said catalyst so that molten sulfur proceeds downwardly toward said upper end portion of the bed;
c) maintaining at least a portion of said bed of said catalyst at one or more temperatures in the range of about 450 to about 700° C. so that (i) said molten sulfur is vaporized at and/or in proximity to the upper end portion of said fixed bed of catalyst and (ii) gaseous sulfur dioxide is converted within said fixed bed in the presence of oxygen and/or air into gaseous sulfur trioxide as a component of said second gaseous stream; and
d) maintaining the proportions of said first gaseous stream and of said molten sulfur entering said headspace such that the ratio of moles of molecular oxygen to moles of molten sulfur and sulfur dioxide as fed into said headspace does not exceed the safe operating temperature limit for the reactor or reactors in which the resultant exothermic reactions are taking place;
whereby said second stream is enriched in sulfur trioxide.

11. A process as in claim 10 wherein when the reactor or each reactor of a series of reactors in which said resultant exothermic reactions are taking place has an operating temperature limit of 1200° F. (ca. 649° C.) said ratio in d) does not exceed about 2.0.

12. A process as in claim 11 wherein said ratio is in the range of approximately 1.25 to 1.75.

13. A process as in claim 10 wherein said vanadium-containing catalyst is comprised of a vanadium pentoxide catalyst that oxidizes sulfur dioxide to sulfur trioxide and that releases from said bed a product gaseous stream comprised of sulfur trioxide.

14. A process as in claim 10 wherein said vanadium-containing catalyst is a catalyst comprised of a mixture of complex inorganic salts containing sodium, potassium and vanadium salts on crystalline silica support, or a catalyst including silica as a support within a salt mixture comprised of potassium and/or cesium sulfates, and vanadium sulfates coated on the solid silica support, said catalyst being a catalyst that oxidizes sulfur dioxide to sulfur trioxide and that releases from said bed a product gaseous stream comprised of sulfur trioxide.

15.-16. (canceled)

17. A process as in claim 10 wherein said headspace provides a zone in which at least a portion of the vaporized sulfur can intermix with said first gaseous stream, and wherein said one or more temperatures are in the range of about 450 to about 600° C.

18. A process of forming sulfur trioxide from sulfur dioxide, which process comprises:
a) passing a first gaseous stream comprised of sulfur dioxide, sulfur trioxide, and oxygen and/or air downwardly into an upstanding reactor containing a fixed bed of a vanadium-containing catalyst that oxidizes sulfur dioxide to sulfur trioxide so that said first gaseous stream proceeds downwardly into a headspace above said fixed bed of catalyst and a gaseous stream from said headspace proceeds downwardly into the fixed bed of said catalyst, said catalyst bed having an upper end portion and a lower end portion;
b) introducing into said first gaseous stream molten sulfur above the upper end portion of said fixed bed of said catalyst;
c) maintaining at least a portion of said bed of said catalyst at one or more temperatures in the range of about 450 to about 700° C. so that said molten sulfur is vaporized at and/or in proximity to the upper end portion of said fixed bed of catalyst and vapors so formed are carried into said fixed bed of catalyst such that a second gaseous stream exits from a lower portion of said fixed bed of catalyst, said second stream containing a higher amount of sulfur trioxide than would be formed in the same reactor with the same catalyst under the same operating conditions except that no sulfur is introduced into said first gaseous stream.

19. A process as in claim 18 wherein said vanadium-containing catalyst is comprised of a vanadium pentoxide catalyst that oxidizes sulfur dioxide to sulfur trioxide and that releases from said bed a product gaseous stream comprised of sulfur trioxide.

20. A process as in claim 18 wherein said vanadium-containing catalyst is comprised of a mixture of complex inorganic salts containing sodium, potassium and vanadium salts on crystalline silica support, or a catalyst including silica as a support within a salt mixture comprised of potassium and/or cesium sulfates, and vanadium sulfates coated on the solid silica support, said catalyst being a catalyst that oxidizes sulfur dioxide to sulfur trioxide and that releases from said bed a product gaseous stream comprised of sulfur trioxide.

21. (canceled)

22. A process as in claim 18 wherein said headspace provides a zone in which at least a portion of the vaporized sulfur can intermix with said first gaseous stream, and wherein said catalyst is maintained at one or more temperatures in the range of about 450 to about 600° C.

23. (canceled)

24. A process of brominating at least one strongly deactivated aromatic compound having one or more substituents with Hammett σₚ>0.2, which process comprises:
A) brominating said compound in an activating solvent comprised of concentrated oleum to cause SO₃ to oxidize hydrogen bromide formed as a coproduct into bromine with the formation of sulfur dioxide as a coproduct;
B) recovering from the bromination in A) (i) a gaseous mixture of sulfur dioxide along with some sulfur trioxide and (ii) a less concentrated oleum;
C) forming a gaseous stream comprised of sulfur dioxide and sulfur trioxide from B), and introducing oxygen and/or air into said stream to form a first gaseous stream comprised of sulfur dioxide, sulfur trioxide, and oxygen and/or air;
D) propelling at least a portion of said first gaseous stream so that it will enter a reactor containing a fixed bed of a vanadium-containing catalyst that oxidizes sulfur dioxide to sulfur trioxide, and introducing molten sulfur into said stream so that (i) vaporized sulfur is formed in the vicinity of the upstream portion of said catalyst bed and (ii) vaporized sulfur together with sulfur dioxide, sulfur trioxide, and oxygen and/or air are transported into said catalyst bed;
E) maintaining the catalyst in said fixed bed at one or more temperatures in the range of about 450 to about 700° C. so that said molten sulfur is vaporized at or in proximity to the upper end portion of said fixed bed of catalyst and vapors so formed are carried into said fixed bed of catalyst such that a second stream enriched in sulfur trioxide emanates from the downstream portion of said catalyst bed; and
F) mixing said second stream with less concentrated oleum recovered in B) to form concentrated oleum for use in A).

25. A process as in claim 24 wherein said fixed bed of a vanadium-containing catalyst is a fixed bed of (i) a vanadium pentoxide catalyst, (ii) a mixture of complex inorganic salts containing sodium, potassium and vanadium salts on crystalline silica support, or (iii) a catalyst including silica as a support within a salt mixture comprised of potassium and/or cesium sulfates, and vanadium sulfates coated on the solid silica support, that oxidizes sulfur dioxide to sulfur trioxide and that releases therefrom a product gaseous stream comprised of sulfur trioxide.

26. A process as in claim 25 wherein said fixed bed of (i), (ii), or (iii) is in an upstanding reactor, wherein the first gaseous stream in D) enters the reactor at its upper end portion above said fixed bed of (i), (ii), or (iii), wherein molten sulfur that is introduced into said stream in D) is introduced into the reactor above said fixed bed of (i), (ii), or (iii), and wherein said second stream emanates from a lower end portion of said fixed bed of (i), (ii), or (iii).

27. A process as in claim 24 wherein said one or more temperatures are in the range of about 450 to about 600° C.

28. A process as in any of claims 24-27 wherein said strongly deactivated aromatic compound is an aromatic anhydride.

29. A process as in claim 28 wherein the bromination in A) is conducted using elemental bromine.

30. A process as in any of claims 24-27 wherein said strongly deactivated aromatic compound is phthalic anhydride.

31. A process as in claim 30 wherein the bromination in A) is conducted using elemental bromine.

32. A process as in claim 24 wherein said reactor containing a fixed bed of a vanadium-containing catalyst includes a headspace above said fixed bed of vanadium-containing catalyst.

33. In the bromination of phthalic anhydride with bromine in a reaction medium comprised of a concentrated oleum and wherein a less concentrated oleum is formed during the bromination, the improvement which comprises mixing at least a portion of said less concentrated oleum with sulfur trioxide formed by a process as in any of claims 1, 5, 10, 11, 12, 13, 14, or 18 to form a concentrated oleum, and using at least a portion of such concentrated oleum in forming reaction medium for bromination of phthalic anhydride with bromine.

34. In a process wherein a first gaseous stream comprised of sulfur dioxide, sulfur trioxide and oxygen and/or air is passed through and in contact with a bed of a vanadium-containing catalyst that oxidizes sulfur dioxide to sulfur trioxide and that releases therefrom a second gaseous stream comprised of sulfur trioxide, the improvement which comprises introducing a gaseous stream comprised of sulfur dioxide formed by uncatalyzed oxidation of sulfur by sulfur trioxide and air, oxygen, or air and oxygen into said first gaseous stream so that the resultant mixture passes through and comes into contact with at least a substantial portion of the catalyst bed, and maintaining the catalyst bed at one or more temperatures in the range of about 450 to about 700° C. so that the second gaseous stream released from the downstream end of said catalyst bed has an enriched content of sulfur trioxide, said vanadium-containing catalyst being a fixed bed of (i) a vanadium pentoxide catalyst, (ii) a mixture of complex inorganic salts containing sodium, potassium and vanadium salts on crystalline silica support, or (iii) a catalyst including silica as a support within a salt mixture comprised of potassium and/or cesium sulfates, and vanadium sulfates coated on the solid silica support.

35. A process of forming sulfur trioxide from sulfur dioxide, which process comprises:
a) passing a first gaseous stream comprised of sulfur dioxide, sulfur trioxide, and oxygen and/or air into a bed of a cesium-promoted vanadium-containing catalyst that oxidizes sulfur dioxide to sulfur trioxide and that releases therefrom a second gaseous stream comprised of sulfur trioxide;
b) providing vaporized sulfur in said first gaseous stream so that the resultant mixture passes into the catalyst bed, and maintaining at least a portion of the catalyst bed at one or more temperatures in the range of about 390 to about 410° C. so that the second gaseous stream released from the downstream end of said catalyst bed has an enriched content of sulfur trioxide.
